(19) United States
(12) Patent Application Publication
(10) Pub. No.: US 2010/0028342 A1
(43) Pub. Date: Feb. 4, 2010

(54) INSULIN-LIKE GROWTH FACTOR-1 RECEPTOR ANTAGONISTS FOR MODULATION OF WEIGHT AND LIPOSITY

(76) Inventor: James R. Tonra, Skillman, NJ (US)

Correspondence Address: ELI LILLY & COMPANY, PATENT DIVISION, P.O. BOX 6288, INDIANAPOLIS, IN, 46206-6288, US

(21) Appl. No.: 12/514,809

(22) PCT Filed: Nov. 29, 2007
(86) PCT No.: PCT/US07/85844

§ 371 Date: May 14, 2009

Related U.S. Application Data
(60) Provisional application No. 60/861,827, filed on Nov. 29, 2006.

(51) Int. Cl. A61K 39/295

(52) U.S. Cl. 424/133.1; 424/172.1

(57) ABSTRACT

The invention is directed to the use of insulin-like growth factor receptor antagonists for treatment of obesity. The IGF-IR antagonists are administered alone or in combination with other anti-obesity drugs.

### CROSS-REFERENCE

This application claims the benefit of U.S. Provisional Application Ser. No. 60/861,827, filed Nov. 29, 2006.

### FIELD OF THE INVENTION

The present invention relates to the use of insulin-like growth factor receptor antagonists for weight maintenance, weight reduction, and treatment of obesity.

### BACKGROUND

Obesity is at epidemic proportions, with greater than 1.1 billion overweight adults worldwide, 312 million of which are considered obese (Haslam, D. W., et al., Lancet 366:1197-1209 (2005)). In the year 2000, 100,000-300,000 deaths in the United States were attributable to obesity. The obesity related comorbidities contributing towards the increased risk of death include ischaemic heart disease, hypertension, stroke, diabetes mellitus, osteoarthritis, and cancer (Haslam et al.). The current status of the obesity epidemic is not due to a lack of effort in treating patients. Billions of dollars are spent every year to induce weight loss in obese patients. These efforts have in fact resulted in weight loss in many obese patients, but inevitably the great majority of patients regain the weight (Goodrick, G. K., et al. J Am Diet Assoc. 91:1243-1247 (1991); Weinsier, R. L., et al. Am J Clin Nutr. 72:1088-1094 (2000)).

One of the physiological pathways thought to be important in obesity is the growth hormone-insulin like growth factor-1 (GH/IGF-I) axis. The human endocrine system is organized into axes serving different functions. The GH/IGF-I axis is critical for normal maturational growth and development (Woods, K. A., et al., N Engl J. Med. 335:1363-1367 (1996); Laron Z., J Clin Endocrinol Metab. 84:4397-4404 (1999)), and is also thought to potentially have a role in regulating metabolism (Franco C, et al., J Clin Endocrinol Metab. 90:1466-1474 (2005); Yakar, S., et al., Pediatr Nephrol, 20:251-254 (2005)). This axis is regulated by factors including stress, exercise, nutrition, and sleep. Neurons in the hypothalamus of the brain responding to these factors regulate growth hormone secretion by somatotroph cells in the pituitary (Mullis P E., Eur J Endocrinol. 152:11-31 (2005)). GH secretion can be increased following release of growth hormone-releasing hormone (GHRH) by hypothalamic neurons, or decreased following release of somatostatin. Growth hormone released from the pituitary can have direct effects on tissues expressing its receptor, or indirect effects following GH induced IGF-I release by the liver. GH and IGF-I exert negative feedback on the axis to regulate the pattern of activity in the GH/IGF-I axis.

While the importance of the GH/IGF-I axis in developmental growth is clear, the role of this axis in adults is less well understood. As with many hormones and growth factors, GH and IGF-I secretion are reduced with ageing (Rosen, C. J., et al, J Clin Endocrinol Met. 82:3919-3922(1997); Toogood, A. A., et al., Horm Res. 60 (Suppl 1):105-111 (2003)), potentially reflecting reduced growth. However, there is evidence indicating a potential role for growth hormone and IGF-I in metabolic functions such as increasing insulin sensitivity and decreasing the body fat/muscle ratio.

In obese patients growth hormone release is significantly reduced and IGF-I levels are reduced relative to normal (Johannsson, G., et al., J Clin Endocrinol Met 82:727-734 (1997)). Due to the fact that obese patients are insulin resistant and have a high body fat/muscle ratio, administering exogenous growth hormone or IGF-I to these patients, has been proposed as a treatment for obesity or its comorbidities (Johannsson et al., Endocrinology 142:3964-3973 (2002)). Exogenous growth hormone has been tested in patients, reducing total body fat in obese patients, with no effect on blood glucose or serum insulin (Johannsson et al.). Exogenous IGF-I has also been tested in patients, increasing insulin sensitivity and decreasing glucose inseverely insulin resistant patients. Despite positive results, the development of the strategy of increasing the activity of the GH/IGF-I axis with exogenous growth factors for the treatment of obesity and its comorbidities has been hindered by the finding of a positive correlation between IGF-1 levels and cancer risk (Jerome, L., et al., Endocr Relat Cancer 10:561-578 (2003)).

The insulin-like growth factor receptor (IGF-IR) is a ubiquitous transmembrane tyrosine kinase receptor that is essential for normal fetal and post-natal growth and development. IGF-IR can stimulate cell proliferation, cell differentiation, changes in cell size, and protect cells from apoptosis. It has also been considered to be quasi-obligatory for cell transformation (reviewed in Adams et al., Cell. Mol. Life. Sci. 57:1050-93 (2000); Baserga, Oncogene 19:5574-81 (2000)). The IGF-IR is located on the cell surface of most cell types and serves as the signaling molecule for growth factors IGF-I and IGF-II (collectively termed henceforth IGFs). IGF-IR also binds insulin, albeit at three orders of magnitude lower affinity than it binds to IGFs. IGF-IR is a pre-formed hetero-tetramer containing two alpha and two beta chains covalently linked by disulfide bonds. The receptor subunits are synthesized as part of a single polypeptide chain of 180 kd, which is then proteolytically processed into alpha (130 kd) and beta (95 kd) subunits. The entire alpha chain is extracellular and contains the site for ligand binding. The beta chain possesses the transmembrane domain, the tyrosine kinase domain, and a C-terminal extension that is necessary for cell differentiation and transformation, but is dispensable for mitogen signaling and protection from apoptosis.

IGF-IR is highly similar to the insulin receptor (IR), particularly within the beta chain sequence (70% homology). Because of this homology, hybrid receptors containing one IR dimer and one IGF-IR dimer can form (Pandini et al., Clin. Canc. Res. 5:1935-19 (1999)). The formation of hybrids occurs in both normal and transformed cells and the hybrid content is dependent upon the concentration of the two homodimer receptors (IR and IGF-IR) within the cell. In one study of 39 breast cancer specimens, although both IR and IGF-IR were over-expressed in all tumor samples, hybrid receptor content consistently exceeded the levels of both homo-receptors by approximately 3-fold (Pandini et al., Clin. Canc. Res. 5:1935-44 (1999)). Although hybrid receptors are composed of IR and IGF-IR pairs, the hybrids bind selectively to IGFs, with affinity similar to that of IGF-IR, and only weakly bind insulin (Siddle and Soos, The IGF System. Humana Press, pp. 199-225. 1999). These hybrids therefore can bind IGFs and transduce signals in both normal and transformed cells.

Endocrine expression of IGF-I is regulated primarily by growth hormone. IGF-I is produced primarily in the liver, but recent evidence suggests that many other, tissue types are also capable of expressing IGF-I. This ligand is therefore subject to endocrine and paracrine regulation, and is also produced by many types of tumor cells (Yu, H. and Rohan, J., J. Natl. Cancer Inst. 92:1472-89 (2000)).

Upon binding of ligand (IGFs), the IGF-IR undergoes autophosphorylation at conserved tyrosine residues within the catalytic domain of the beta chain. Subsequent phosphorylation of additional tyrosine residues within the beta chain provides docking sites for the recruitment of downstream molecules critical to the signaling cascade. The principle pathways for transduction of the IGF signal are mitogen activated protein kinase (MAPK) and phosphatidylinositol 3-kinase (PI3K) (reviewed in Blakesley et al., In: The IGF System. Humana Press. 143-163 (1999)). The MAPK pathway is primarily responsible for the mitogenic signal elicited following IGFs stimulation and PI3K is responsible for the IGF-dependent induction of anti-apoptotic or survival processes.

A key role of IGF-IR signaling is its anti-apoptotic or survival function. Activated IGF-IR signals PI3K and downstream phosphorylation of Akt, or protein kinase B. Akt can effectively block, through phosphorylation, molecules such as BAD, which are essential for the initiation of programmed cell death, and inhibit initiation of apoptosis (Datta et al., Cell 91:231-41 (1997)). Apoptosis is an important cellular mechanism that is critical to normal developmental processes (Oppenheim, Annu. Rev. Neurosci. 14:453-501 (1991)). It is a key mechanism for effecting the elimination of severely damaged cells and reducing the potential persistence of mutagenic lesions that may promote tumorigenesis. To this end, it has been demonstrated that activation of IGF signaling can promote the formation of spontaneous tumors in a mouse transgenic model (DiGiovanni et al., Cancer Res. 60:1561-70 (2000)). Furthermore, IGF over-expression can rescue cells from chemotherapy induced cell death and may be an important factor in tumor cell drug resistance (Gooch et al., Breast Cancer Res. Treat. 56:1-10 (1999)). Consequently, down modulation of the IGF signaling pathway has been shown to increase the sensitivity of tumor cells to chemotherapeutic agents (Benini et al., Clinical Cancer Res. 7:1790-97 (2001)).

A large number of research and clinical studies have implicated the IGF-IR and its ligands (IGFs) in the development, maintenance, and progression of cancer. In tumor cells, over-expression of the receptor, often in concert with over-expression of IGF ligands, leads/to potentiation of these signals and, as a result; enhanced cell proliferation and survival. Activation of the IGF system has also been implicated in several pathological conditions besides cancer, including acromegaly (Drange and Melmed. In: The IGF System. Humana Press. 699-720 (1999)), retinal neovascularization (Smith et al., Nature Med. 12:1390-95 (1999)), and psoriasis (Wraight et al., Nature Biotech. 18:521-26 (2000)). In the latter study, an antisense oligonucleotide preparation targeting the IGF-IR was effective in significantly inhibiting the hyperproliferation of epidermal cells in human psoriatic skin grafts in a mouse model, suggesting that anti-IGF-IR therapies may be an effective treatment for this chronic disorder.

A variety of strategies have been developed to inhibit the IGF-IR signaling pathway in cells. Antisense oligonucleotides have been effective in vitro and in experimental mouse models, as shown above for psoriasis. Several small molecule inhibitors of IGF-IR have been developed. In addition, inhibitory peptides targeting the IGF-IR have been generated that possess anti-proliferative activity in vitro and in vivo (Pietrzkowski et al., Cancer Res. 52:6447-51 (1992); Haylor et al., J. Am. Soc. Nephrol. 11:2027-35 (2000)). A synthetic peptide sequence from the C-terminus of IGF-IR has been shown to induce apoptosis and significantly inhibit tumor growth (Reiss et al., J. Cell. Phys. 181:124-35 (1999)). Several dominant-negative mutants of the IGF-IR have also been generated which, upon over-expression in tumor cell lines, compete with wild-type IGF-IR for ligand and effectively inhibit tumor cell growth in vitro and in vivo (Scotlandi et al., Int. J. Cancer 101:11-6 (2002); Seely et al., BMC Cancer 2:15 (2002)). Additionally, a soluble form of the IGF-IR has also been demonstrated to inhibit tumor growth in vivo (D'Ambrosio et al., Cancer Res. 56:4013-20 (1996)). Antibodies directed against the human IGF-IR have also been shown to inhibit tumor cell proliferation in vitro and tumorigenesis in vivo including cell lines derived from breast cancer (Artega and Osborne, Cancer Res. 49:6237-41 (1989)), Ewing's osteosarcoma (Scotlandi et al., Cancer Res. 58:4127-31 (1998)), and melanoma (Furlanetto et al., Cancer Res. 53:2522-26 (1993)). Antibodies are attractive therapeutics chiefly because they 1) can possess high selectivity for a particular protein antigen, 2) are capable of exhibiting high affinity binding to the antigen, 3) possess long half-lives in vivo, and, since they are natural immune products, should 4) exhibit low in vivo toxicity (Park and Smolen. In: Advances in Protein Chemistry. Academic Press. pp:360-421 (2001)). Following repeated application, antibodies derived from non-human sources, e.g., mouse, may effect a directed immune response against the therapeutic antibody, thereby neutralizing the antibody's effectiveness. Fully human antibodies offer the greatest potential for success as human therapeutics since they would likely be less immunogenic than murine or chimeric antibodies in humans, similar to naturally occurring immuno-responsive antibodies.

### SUMMARY OF THE INVENTION

The present invention provides novel therapeutic methods for modulating body weight. Further, the invention provides compositions for use in such therapies. In contrast with current dogma (Johannsson G., et al.) and efforts in the literature and in the clinic focused on activating the GH/IGF-I axis, the present invention centers on blocking the IGF-IR signaling for the treatment of obesity and its comorbidities.

Thus, the invention provides for methods of modulating body weight in mammals, e.g., humans, in a process that includes blocking IGF-IR signaling by administering an insulin-like growth factor receptor (IGF-IR) antagonist to a mammal in need thereof. The modulating of body weight can result in loss of body weight, maintaining body weight, or minimizing increases in body weight following weight loss in said mammal.

According to the present invention, antagonists to the GH/IGF-I axis, particularly IGF-IR antagonists, are used to effect loss of body weight, to maintain body weight, or to minimize or prevent increases in body weight following weight loss. The IGF-IR antagonists are also used to modulate body composition (e.g., to reduce percent body fat). The invention provides methods and compositions for modulating IGF-IR mediated signal transduction that are effective to modulate the body weight or composition of an individual, and are particularly advantageous for treatment of an overweight or obese individual.

IGF-IR antagonists are molecules that block, modulate or impede the signaling mediated by IGF-IR, and include, but are not limited to, antibodies, small molecules, proteins, polypeptides, IGF mimetics, antisense oligodeoxynucleotides, antisense RNAs, small inhibitory RNAs, triple helix forming nucleic acids, dominant negative mutants, and soluble receptor expression.

In one embodiment of the invention, the IGF-IR antagonist binds to IGF-IR and blocks ligand binding. In another embodiment of the invention, the IGF-IR antagonist binds to IGF-IR and promotes reduction in IGF-IR surface receptor. In yet another embodiment of the invention, the IGF-IR antagonist binds to IGF-IR and inhibits IGF-IR mediated signal transduction.

In an embodiment of the invention, the IGF-IR antagonist is an antibody. In certain embodiments, the IGF-IR antagonists are antibodies that bind to IGF-IR with a K_{d} that is less than about 10⁻⁹ M⁻¹ or less than about 10⁻¹⁰ M⁻¹ or less than about 3×10⁻¹⁰ M⁻¹. Non-limiting examples of anti-IGF-IR antibodies include A12 and 2F8 (described below), and antibodies that compete with A12 and/or 2F8 for binding to IGF-IR. Antibodies that can be used according to the invention include chimeric and humanized antibodies. In a preferred embodiment, the antibody is human. In another embodiment, the IGF-IR antagonist is a mimetic of an IGF-IR ligand that binds to, but does not activate, the receptor. In yet another embodiment, the IGF-IR antagonist is a small molecule (e.g., an element of a combinatorial chemistry library or a low molecular weight natural or synthetic product or metabolite) that binds to the ligand binding domain of IGF-IR and blocks binding of an IGF-IR ligand. In another embodiment of the invention, the IGF-IR antagonist blocks interaction of IGF-IR with its substrate IRS-1.

### BRIEF DESCRIPTION OF THE FIGURES

FIG. 1 shows binding and blocking of anti-IGF-IR antibodies. (A) Binding, of A12 and 2F8 on immobilized recombinant IGF-IR. (B) Blocking of ¹²⁵I-IGF-I binding to immobilized IGF-IR by antibodies 2F8 or A12, or ligands IGF-I or IGF-II. (C) Blocking of ¹²⁵I-IGF-I binding to native IGF-IR on MCF7 cells.

FIG. 2 shows inhibition of IGF-IR phosphorylation and IGF-IR mediated signal transduction. (A) Inhibition of IGF-I induced phosphorylation of IGF-IR in MCF7 breast cancer cells by antibodies A12 and 2F8; (B) Inhibition of IGF-I and IGF-II mediated phosphorylation of downstream effector molecules in MCF7 cells by antibody A12. Western blots of MCF7 cell lysates were probed for phosphorylated IRS-1 (pIRS-1), MAPK (pMAPK), and Akt (pAkt).

FIG. 3 shows lack of insulin receptor (IR) binding and blocking activity by A12. (A) Binding of A12 to immobilized IR. Anti-IR antibody 47-9 was used as a positive control. (B) Blocking of ¹²⁵I-insulin binding to immobilized IR by insulin, IGF-I, A12, or anti-IR antibody 49-7.

FIG. 4 shows binding of A12 to recombinant mouse and human IGF-IR.

FIG. 5 shows the effect of A12 on body weight in lean Balb/c mice. Female Balb/c mice were treated with A12 at 40 mg/kg, M-W-F, with and without a loading dose of 140 mg/kg. Control mice were treated with human IgG at 40 mg/kg, M-W-F, or TBS at 0.5 ml/dose, M-W-F. Mice were started on treatment at an immature (A) or more mature (B) body weight. Treatments were stopped at the indicated times. Mean body weight±SEM is plotted (n=5 per group).

FIG. 6 depicts food intake in obese ob/ob mice. Ob/ob mice were left untreated or underwent diet restriction through daily feeding of a reduced quantity of rodent chow. Following diet restriction, these mice were treated with human IgG or A12 at 30 mg/kg, on a Tuesday, Friday schedule. Mean food intake SEM is plotted (n=7 per group). * indicates time points at which fresh food was added to the ad libitum fed mice, which led to transient spikes in food intake.

FIG. 7 shows the effect of A12 on body weight in obese ob/ob mice. Ob/ob mice were left untreated or underwent diet restriction through daily feeding of a reduced quantity of rodent chow. Following diet restriction, these mice were fed ad libitum, starting five hours after the start of i.p. treatment with human IgG or A12 at 30 mg/kg, on a Tuesday, Friday schedule. The final treatment was administered 25 days after the start of diet restriction. Mean body weight SEM is plotted (h=7 per group).

FIG. 8 shows the effect of A12 on body weight of obese mice that have been fed a restricted diet and on obese mice fed ad libitum. Diet restricted mice were fed reduced amounts of food for 13 days, then fed ad libitum starting three hours after the start of i.p. treatment with human IgG at 30 mg/kg or A12 at 3, 10, or 30 mg/kg. In addition, mice that had not been diet restricted were treated with 30 mg/kg of A12 on the same schedule as the diet restricted mice. Mean body weight SEM is plotted (n=4-12 per group).

### DETAILED DESCRIPTION OF THE INVENTION

The present invention relates to the use of IGF-IR antagonists for reducing body weight as well as for maintaining body weight and reducing weight gain. In certain embodiments of the invention, the IGF-IR antagonists are used to treat individuals that are overweight or obese.

Normal weight varies with sex, height, and age, and the standards that define an individual as normal, overweight, or obese have changed over time. Further, body composition parameters, such as percent fat weight and lean body weight, are significant determinants of disease risk. Accordingly, it is useful to employ specific measures for overweight and obesity.

One way to measure body fat content is by densitometry. Since fat tissue has a lower density than muscles and bones, it is possible to estimate a person's fat content by weighing the person underwater in order to obtain the average density. Body fat percentage can then be calculated based on average density. One commonly used formula is percent body fat=(4.95/ρ−4.50)×100. (Siri, W. E., 1961, in Techniques for Measuring Body Composition. J. Brozekand A. Henschel, ed. National Academy of Sciences, Washington, D.C., pp. 223-244.). Total fat mass can be calculated as total body mass×percent body fat. Lean body mass (LBM) is the difference between total body mass and fat mass.

Another non-invasive approach to assess body fat is dual-energy X-ray absorptiometry (DEXA). DEXA can be used to estimate whole-body fat as well as fat in specific anatomical regions. A simple but less reliable test for measuring body fat is the skinfold, test, whereby a pinch of skin is measured by calipers, at several standardized points on the body to determine the thickness of the subcutaneous fat layer.

While body composition (i.e., adiposity) is more closely related to disease and mortality risks than body weight, an index of body mass corrected for height can give a good approximation of fat content for most individuals. Body mass index (BMI) is an easily determined and relatively reliable measurement. If weight is measured in pounds and height In inches, the BMI. (units=kg/m²) is calculated as (weight (lb)/height (in)²)×703. If weight is measured in kilograms and height in meters, the formula is BMI (units=kg/m²)=weight (kg)/height (m)². This index gives body mass corrected for height for a wide range of heights and is a good approximate estimate of the fat content of the body. The current diagnostic criteria of obesity for adults are based on epidemiologic data concerning risks of disease and mortality. Obesity is currently indicated by a BMI≧30 kg/m². Morbid obesity correlates with a BMI of ≧40 kg/m² or with being 100 pounds overweight. Morbidity and mortality increase gradually with BMI, and there is also increased risk associated with a BMI under 30 kg/m². Accordingly, a BMI≧25 and less than 30 kg/m² is considered diagnostic of “overweight.”

The correlation between the BMI and body fatness is fairly strong, but varies, by sex, race, age and conditioning. Thus, it is important to remember that BMI is only one factor related to likelihood of developing overweight- or obesity-related diseases. Another important predictors is an individual's waist circumference (because abdominal fat is a predictor of risk for obesity-related diseases).

The present invention is used to reduce or to prevent or to minimize the increase of fat mass (or percent body fat) or BMI in a subject. In certain embodiments of the invention, die body fat percent of a subject to be treated is equal to or greater than about 10, or equal to or greater than about 20, or equal to or greater than about 30. In other embodiments of the invention, the BMI of a subject to be treated is equal to or greater than about 20 kg/m², or equal to or greater than about 25 kg/m², or equal to or greater than about 30 kg/m², or equal to or greater than about 40 kg/m².

IGF-IR antagonists include any substances that inhibit IGF-IR mediated signal transduction. Accordingly, IGF-IR antagonists include extracellular antagonists and intracellular antagonists. Extracellular antagonists are typically substances that reduce or block receptor-ligand interactions. Extracellular antagonists can also function to down regulate cell surface receptor. Extracellular antagonists include antibodies and other proteins or polypeptides that bind to IGF-IR, and antibodies or other proteins or polypeptides specific for an IGF-IR ligand.

Naturally occurring antibodies typically have two identical heavy chains and two identical light chains, with each light chain covalently linked to a heavy chain by an interchain disulfide bond and multiple disulfide bonds further link the two heavy chains to one another. Individual chains can fold into domains having similar sizes (110-125 amino acids) and structures, but different functions. The light chain can comprise one variable domain (V_{L}) and/or one constant domain (C_{L}). The heavy chain can also comprise one variable domain (V_{H}) and/or, depending on the class or isotype of antibody, three or four constant domains (C_{H}1, C_{H}2, C_{H}3 and C_{H}4). In humans, the isotypes are IgA, IgD, IgE, IgG, and IgM, with IgA and IgG further subdivided into subclasses or subtypes (IgA₁₋₂ and IgG₁₋₄).

Generally, the variable domains show considerable amino acid sequence variability from one antibody to the next, particularly at the location of the antigen-binding site. Three regions, called hypervariable or complementarity-determining regions (CDRs), are found in each of V_{L} and V_{H}, which are supported by less variable regions called frameworks (FWs).

The portion of an antibody consisting of V_{L} and V_{H} domains is designated Fv (Fragment variable) and constitutes the antigen-binding site. Single chain Fv (scFv) is an antibody fragment containing a V_{L} domain, and a V_{H} domain on one polypeptide chain, wherein the N terminus of one domain and the C terminus of the other domain are joined by a flexible linker (see, e.g., U.S. Pat. No. 4,946,778 (Ladner et al.); WO 88/09344, (Huston et al.). WO 92/01047 (McCafferty et al.) describes the display of scFv fragments on the surface of soluble recombinant genetic display packages, such as bacteriophage.

The peptide linkers used to produce the single chain antibodies can be flexible, peptides selected to assure that the proper three-dimensional folding and association of the V_{L} and V_{H} domains occurs. The linker is generally 10 to 50 amino acid residues. Preferably, the linker is 10 to 30 amino acid residues. More preferably the linker is 12 to 30 amino acid residues. Most preferably is a linker of 15 to 25 amino acid residues. A non-limiting example of such a linker peptides is (Gly-Gly-Gly-Gly-Ser)₃ (SEQ ID NO:33).

Fab (Fragment, antigen binding) refers to the fragments of the antibody consisting of V_{L}-C_{L} and V_{H}-C_{H}1 domains. Such a fragment generated by digestion of a whole antibody with papain does not retain the antibody hinge region by which two heavy chains are normally linked. The fragment is monovalent and simply referred to as Fab. Alternatively, digestion with pepsin results in a fragment that retains the hinge region. Such a fragment with intact interchain disulfide bonds linking two heavy chains is divalent and is referred to as F(ab′)₂. A monovalent Fab′ results when the disulfide bonds of an F(ab′)₂ are reduced (and the heavy chains are separated. Because they are divalent, intact antibodies and F(ab′)₂ fragments have higher avidity for antigen that the monovalent Fab or Fab′ fragments. WO 92/01047 (McCafferty et al.) describes the display of Fab fragments on the surface of soluble recombinant genetic display packages, such as bacteriophage.

Fc (Fragment crystallization) is the designation for the portion or fragment of an antibody that consists of paired heavy chain constant domains. In an IgG antibody, for example, the Fc consists of heavy chain C_{H}2 and C_{H}3 domains. The Fc of an IgA or an IgM antibody further comprises a C_{H}4 domain. The Fc is associated with Fc receptor binding, activation of complement-mediated cytotoxicity and antibody-dependent cellular-cytotoxicity (ADCC). For antibodies such as IgA and IgM, which are complexes of multiple IgG like proteins, complex formation requires Fc constant domains.

Finally, the hinge region separates the Fab and Fc portions of the antibody, providing for mobility of Fabs relative to each other and relative to Fc, and provides disulfide bonds for covalent linkage of the two heavy chains.

Antibody formats have been developed which retain binding specificity, but have other characteristics that may be desirable, including for example, bispecificity, multivalence (more than two binding sites), and compact size (e.g., binding domains alone).

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies; are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one V_{H} and one V_{L} domain covalently linked by a first peptide linker, can be covalently linked by at least one or more peptide linker to form a multivalent single chain antibodies, which can be monospecific or multispecific. Each chain of a multivalent, single chain antibody includes, a variable light chain fragment and a variable heavy chain fragment, and is linked by a peptide linker to at least one other chain. The peptide linker is generally composed of at least fifteen amino acid residues. The maximum number of amino acid residues is about one hundred.

Two single chain antibodies can be combined to form a diabody, also known as a bivalent dimer. Diabodies have two chains and two binding sites, and can be monospecific or bispecific. Each chain of the diabody includes a V_{H} domain connected to a V_{L} domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a V_{L} or V_{H} domain directly fused to the carboxyl terminus of a V_{L} or V_{H} domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Thus, antibodies of the invention and fragments thereof include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with antigens.

The antibodies of the present invention and particularly the variable domains thereof may be obtained by methods known in the art. These methods include, for example, the immunological method described by Kohler and Milstein, Nature 256:495-497 (1975) and Campbell, Monoclonal Antibody Technology, The Production and Characterization of Rodent and Human Hybridomas, Burdon et al., Eds., Laboratory Techniques in Biochemistry and Molecular Biology, Volume 13, Elsevier Science Publishers, Amsterdam (1985); as well as by the recombinant DNA methods such as described by Huse et al., Science 246, 1275-81 (1989). The antibodies can also be obtained from phage display libraries bearing combinations of V_{H} and V_{L} domains in the form of scFv or Fab. The V_{H} and V_{L} domains can be encoded by nucleotides that are synthetic, partially synthetic, or naturally derived. In certain embodiments, phage display libraries bearing human antibody fragments can be preferred. Other sources of human antibodies are transgenic mice engineered to express human immunoglobulin genes.

Antibody fragments can be produced by cleaving a whole antibody, or by expressing DNA that encodes the fragment. Fragments of antibodies may be prepared by methods described by Lamoyi et al., J. Immunol. Methods 56:235-243 (1983) and by Parham, J. Immunol. 131:2895-2902 (1983). Such fragments may contain one or both Fab fragments or the F(ab′)₂ fragment. Such fragments may also contain single-chain fragment variable region antibodies, i.e. scFv, dibodies, or other antibody fragments. Methods of producing such functional equivalents are disclosed in PCT Application WO 93/21319, European Patent Application No. 239,400; PGT Application WO 89/09622; European Patent Application 338,745; and European Patent Application EP 332,424.

The antibodies, or fragments thereof, of the present invention are specific for IGF-IR. Antibody specificity refers to selective recognition of the antibody for a particular epitope of an antigen. Antibodies, or fragments thereof, of the present invention, for example, can be monospecific or bispecific. Bispecific antibodies (BsAbs) are antibodies that have two different antigen-binding specificities or sites. Where an antibody has more than one specificity, the recognized epitopes can be associated with a single antigen or with more than one antigen. Thus, the present invention provides bispecific antibodies, or fragments thereof, that bind to two different antigens, with at least one specificity for IGF-IR.

Specificity of the present antibodies, or fragments thereof, for IGF-IR can be determined based on affinity and/or avidity. Affinity, represented by the equilibrium constant for the dissociation of an antigen with an antibody (Kd), measures the binding strength between an antigenic determinant and an antibody-binding site. Avidity is the measure, of the strength of binding between an antibody with its antigen. Avidity is related to both the affinity between an epitope with its antigen binding site on the antibody, and the valence of the antibody, which refers to the number of antigen binding sites specific for a particular epitope. Antibodies typically bind with a dissociation constant (Kd) of 10⁻⁵ to 10⁻¹¹ liters/mol. or better. Any Kd greater than 10⁻⁴ liters/mol is generally considered to indicate nonspecific binding. The lesser the value of the Kd, the stronger the binding strength between an antigenic determinant and the antibody binding site.

Antibodies of the present invention, or fragments thereof, also include those for which binding characteristics have been improved by direct mutation, methods of affinity maturation, phage display, or chain shuffling. Affinity and specificity can be modified or improved by mutating CDR and/or FW residues and screening for antigen binding sites having the desired characteristics (see, e.g., Yang et al., J. Mol. Biol. 254:392-403 (1995)). One way is to randomize individual residues or combinations of residues so that in a population of, otherwise identical antigen binding sites, subsets of from two to twenty amino acids are found at particular positions. Alternatively, mutations can be induced over a range of residues by error prone PCR methods (see, e.g., Hawkins et al., J. Mol. Biol. 226:889-96 (1992)). In another example, phage display vectors containing heavy and light chain variable region genes can be propagated in mutator strains of E. coli (see, e.g., Low et al., J. Mol. Biol. 250:359-68 (1996)). These methods of mutagenesis are illustrative of the many methods known to one of skill in the art.

Conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical substitutions that may be performed for such conservative amino acid substitution may be among the groups of amino acids as follows:

glycine (G), alanine (A), valine (V), leucine (L) and isoleucine (I);

aspartic acid (D) and glutamic acid (E);

alanine (A), serine (S) and threonine (T);

histidine (H), lysine (K) and arginine (R):

asparagine (N) and glutamine (Q);

phenylalanine (F), tyrosine (Y) and tryptophan (W)

Conservative amino acid substitutions can be made in, e.g., regions flanking the hypervariable regions primarily responsible for the selective and/or specific binding characteristics of the molecule, as well as other parts of the molecule, e.g., variable heavy chain cassette.

Each domain of the antibodies of this invention can be a complete antibody with the heavy or light chain variable domain, or it can be a functional equivalent or a mutant or derivative of a naturally-occurring domain, or a synthetic domain constructed, for example, in vitro using a technique such as one described in WO 93/11236 (Griffiths et al.). For instance, it is possible to join together domains corresponding to antibody variable domains, which are missing at least one amino acid. The important characterizing feature is the ability of each domain to associate with a complementary domain to form an antigen-binding site. Accordingly, the terms variable heavy and light chain fragment should not be construed to exclude variants that do not have a material effect on specificity.

In preferred embodiments, the anti-IGF-IR antibodies of the present invention are human antibodies that exhibit one or more of several properties. In one embodiment, the antibodies bind to the external domain of IGF-IR and inhibit binding of IGF-I or IGF-II to IGF-IR. Inhibition can be determined, for example, by a direct binding assay using purified or membrane bound receptor. In this embodiment, the antibodies of the present invention, or fragments thereof, preferably bind IGF-IR at least as strongly as the natural ligands of IGF-IR (IGF-I and IGF-II).

In an embodiment of the invention, the antibodies neutralize IGF-IR, Binding of a ligand, e.g., IGF-I or IGF-II, to an external, extracellular domain of IGF-IR stimulates autophosphorylation of the beta subunit and phosphorylation of IGF-IR substrates, including MAPK, Akt, and IRS-1. Neutralization of IGF-IR includes inhibition, diminution, inactivation and/or disruption of one or more of these activities normally associated with signal transduction. Neutralization of IGF-IR includes inhibition of IGF-IR/IR heterodimers as well as IGF-IR homodimers. Thus, neutralizing IGF-IR has various effects, including, but not limited to, inhibition, diminution, inactivation and/or disruption of growth (proliferation and differentiation), angiogenesis (blood vessel recruitment, invasion, and metastasis), and cell motility and metastasis (cell adhesion and invasiveness).

One measure of IGF-IR neutralization is inhibition of the tyrosine kinase activity of the receptor. Tyrosine kinase inhibition can be determined using well-known methods; for example, by measuring the autophosphorylation level of recombinant kinase receptor, and/or phosphorylation of natural or synthetic substrates. Thus, phosphorylation assays are useful in determining neutralizing: antibodies in the context of the present invention. Phosphorylation can be detected, for example, using ah antibody specific for phosphotyrosine in an ELISA assay or on a western blot. Some assays for tyrosine kinase activity are described in Panek et al., J. Pharmacol. Exp. Thera. 283:1433-44 (1997) and Batley et al., Life Sci. 62:143-50 (1998). Antibodies of the invention cause a decrease in tyrosine phosphorylation of IGF-IR of at least about 75%, preferably at least about 85%, and more preferably at least about 90% in cells that respond to ligand.

Another measure of IGF-IR neutralization is inhibition of phosphorylation of downstream substrates of IGF-IR. Accordingly, the level of phosphorylation of MAPK, Akt, or IRS-1 can be measured. The decrease in substrate phosphorylation is at least about 50%, preferably at least about 65%, more preferably at least about 80%.

In addition, methods for detection of protein expression can be utilized to determine IGF-IR neutralization, wherein the proteins being measured are regulated by IGF-IR tyrosine kinase activity. These methods include immunohistochemistry (IHG) for detection of protein expression, fluorescence in situ hybridization (FISH) for detection of gene amplification, competitive radioligand binding assays, solid matrix blotting techniques, such as Northern and Southern blots, reverse transcriptase polymerase chain reaction (RT-PCR) and ELISA. See, e.g., Grandis et al., Cancer, 78:1284-92 (1996); Shimizu et al., Japan J. Cancer Res., 85:567-71 (1994); Sauter et al., Am. J. Path., 148:1047-53 (1996); Collins, Glia 15:289-96 (1995); Radinsky et al., Clin. Cancer Res. 1:19-31 (1995); Petrides et al., Cancer Res. 50:3934-39 (1990); Hoffmann et al., Anticancer Res. 17:4419-26 (1997); Wikstrand et al., Cancer Res. 55:3140-48 (1995).

In vivo assays can also be utilized to determine IGF-IR neutralization. For example, receptor tyrosine kinase inhibition can be observed by mitogenic assays using cell lines stimulated with receptor ligand in the presence and absence of inhibitor. For example, MCF7 (American Type Culture Collection (ATCC), Rockville, Md.) stimulated with IGF-I or IGF-II can be used to assay IGF-IR inhibition. Another method involves testing for inhibition of growth of IGF-IR-expressing tumor cells or cells transfected to express IGF-IR. Inhibition can also be observed using tumor models, for example, human tumor cells injected into a mouse. The present invention is not limited by any particular mechanism of IGF-IR neutralization.

In an embodiment of the invention, the antibodies down modulate IGF-IR. The amount of IGF-IR present on the surface of a cell depends on receptor protein production, internalization, and degradation. The amount of IGF-IR present on the surface of a cell can be measured indirectly, by detecting internalization of the receptor or a molecule bound to the receptor. For example, receptor internalization can be measured by contacting cells that express IGF-IR with a labeled antibody. Membrane-bound antibody is then stripped, collected and counted. Internalized antibody is determined by lysing the cells and, detecting label in the lysates.

Another way to determine down-modulation is to directly measure the amount of the receptor present on the cell following treatment with an anti-IGF-IR antibody or other substance, for example, by fluorescence-activated cell-sorting analysis of cells stained for surface expression of IGF-IR. Stained cells are incubated at 37° C. and fluorescence intensity measured over time. As a control, part, of die stained population can be incubated at 4° C. (conditions under which receptor internalization is halted). Cell surface IGF-IR can also be detected and measured using a different antibody that is specific for IGF-IR, and that does not block or compete with binding of the antibody being tested. (Burturn, et al. Cancer Res. 63:8912-21 (2003))

Treatment of an IGF-IR expressing cell with an antibody of the invention results in reduction of cell surface IGF-IR. In a preferred embodiment, the reduction is at least about 70%, more preferably at least about 80%, and even more preferably at least about 90% in response to treatment with an antibody of the invention. A significant decrease can be observed in as little as four hours.

Another measure of down-modulation is reduction of the total receptor protein present in a cell, and reflects degradation of internal receptors. Accordingly, treatment of cells (particularly cancer cells) with antibodies of the invention results in a reduction in total cellular IGF-IR. In a preferred embodiment, the reduction is at least about 70%, more preferably at least about 80%, and even more preferably at least about 90%.

In preferred embodiments, the antibodies of the invention bind to IGF-IR with a K_{d} of about 10⁻⁹ M⁻¹ or less, or a K_{d} of about 3×10⁻¹⁰ M⁻¹ or less, or about 1×10⁻¹⁰ M⁻¹ or less, or about 3×10⁻¹¹ M⁻¹ or less.

An example of an antibody or fragments of an antibody suitable for the present invention are human antibodies having one, two, three, four, five, and/or six complementarity determining regions (CDRs) selected from the group consisting of SEQ ID NO:14, SEQ ID NO:16, SEQ ID NO:18, SEQ ID NO:20, SEQ ID NO:22, SEQ ID NO:24, SEQ ID NO:26, SEQ ID NO:28, and SEQ ID NO:30. Preferably, the antibodies (or fragments thereof) of the present invention have CDRs of SEQ ID NO:14, SEQ ID NO:16 and SEQ ID NO:18. Alternatively and also preferably, the present antibodies, or fragments thereof, have CDRs of SEQ ID NO:20, SEQ ID NO:22 and SEQ ID NO:24. Alternatively and also preferably, the present antibodies, or fragments thereof, have CDRs of SEQ ID NO:26, SEQ ID NO:28 and SEQ ID NO:30. The amino acid sequences of the CDRs are set forth below in Table 1.

TABLE 1
Heavy Chain (2F8/A12)

| | | |
|---|---|---|
| CDR1 | SYAIS | SEQ ID NO: 14 |
| CDR2 | GIIPIFGTANYAQKFQG | SEQ ID NO: 16 |
| CDR3 | APLRFLEWSTQDHYYYYYMDV | SEQ ID NO: 18 |

Light Chain (2F8)

| | | |
|---|---|---|
| CDR1 | QGDSLRSYYAS | SEQ ID NO: 20 |
| CDR2 | GKNNRPS | SEQ ID NO: 22 |
| CDR3 | NSRDNSDNRLI | SEQ ID NO: 24 |

Light Chain (A12)

| | | |
|---|---|---|
| CDR1 | QGDSLRSYYAT | SEQ ID NO: 26 |
| CDR2 | GENKRPS | SEQ ID NO: 28 |
| CDR3 | KSRDGSGQHLV | SEQ ID NO: 30 |

In another embodiment, the present antibodies, or fragments thereof, can have a heavy chain variable region of SEQ ID NO:2 and/or a light chain variable region selected from SEQ ID NO:6 or SEQ ID NO:10. A12 is an example of an antibody of the present invention. This antibody has human V_{H} and V_{L} framework regions (FWs) as well as CDRs. The V_{H} variable domain of A12 (SEQ ID NO:2) has three CDRs corresponding to SEQ ID NOS:14, 16, and 18 and the V_{L} domain (SEQ ID NO:10) has three CDRs corresponding to SEQ ID NOS:26, 28, and 30. 2F8 is another example of an antibody of the present invention. This antibody also has human V_{H} and V_{L} framework regions (FWs) and CDRs. The V_{H} variable domain of 2F8 is identical to the V_{H} variable domain of A12. The V_{L} domain of 2F8 (SEQ ID NO:6) has three CDRs corresponding to SEQ ID NOS:20, 22, and 24.

In another embodiment, antibodies of the invention compete for binding to IGF-IR with A12 and/or 2F8. That is, the antibodies bind to the same or similar overlapping epitope.

The present invention also provides isolated polynucleotides encoding the antibodies, or fragments thereof, described previously. The invention includes nucleic acids having a sequence encoding one, two, three, four, five and/or all six CDRs as set forth in Table 2.

TABLE 2
Heavy Chain (2F8/A12)

| | | |
|---|---|---|
| CDR1 | agctatgcta tcagc | SEQ ID NO: 13 |
| CDR2 | gggatcatcc ctatctttgg tacagcaaac | SEQ ID NO: 15 |
| | tacgcacaga agttccaggg c | |
| CDR3 | gcgccattac gatttttgga gtggtccacc | SEQ ID NO: 17 |
| | caagaccact actactacta ctacatg | |
| | gacgtc | |

Light Chain (2F8)

| | | |
|---|---|---|
| CDR1 | caaggagaca gcctcagaag ctattatgca | SEQ ID NO: 19 |
| | agc | |
| CDR2 | ggtaaaaaca accggccctc a | SEQ ID NO: 21 |
| CDR3 | aactcccggg acaacagtga taaccgtctg | SEQ ID NO: 23 |
| | ata | |

Light Chain (A12)

| | | |
|---|---|---|
| CDR1 | caaggagaca gcctcagaag ctattatgca | SEQ ID NO: 25 |
| | acc | |
| CDR2 | ggtgaaaata agcggccctc a | SEQ ID NO: 27 |
| CDR3 | aaatctcggg atggcagtgg tcaacatctg | SEQ ID NO: 29 |
| | gtg | |

DNA encoding human antibodies can be prepared by recombining DNA encoding human constant regions and variable regions, other than the CDRs, derived substantially or exclusively from the corresponding human antibody regions and DNA encoding CDRs derived from a human (e.g., SEQ ID NOs:13, 15, and, 17 for the heavy chain variable domain CDRs and SEQ ID NOs:19, 21, and 23 or SEQ ID NOS:25, 27 and 29 for the light chain variable domain CDRs).

Other suitable sources of DNAs that encode fragments of antibodies include any cell, such as hybridomas and spleen cells, that express the full-length antibody. The fragments may be used by themselves as antibody equivalents, or may be recombined into equivalents, as described above. The DNA recombinations and other techniques described in this section may be carried out by known methods. Other sources of DNAs are single chain antibodies or Tabs produced from a phage display library, as is known in the art.

The present invention also include antibodies with amino acid sequences substantially the same as the amino acid sequence of the variable or hypervariable regions of the full-length anti-IGF-IR antibodies. Substantially the same amino acid sequence is defined herein as a sequence with at least 70%, preferably at least, about 80%, and more preferably at least about 90% homology to another amino acid sequence, as determined by the FAST A search method in accordance with Pearson and Lipman (Proc. Natl. Acad. Sci. USA 85:2444-8 (1998)).

In addition, the present invention provides expression vectors containing the polynucleotide sequences previously described operably linked to an expression sequence, a promoter and an enhancer sequence. A variety of expression vectors for the efficient synthesis of antibody polypeptide in prokaryotic, such as bacteria and eukaryotic systems, including but not limited to yeast and mammalian cell culture systems have been developed. The vectors of the present invention can comprise segments of chromosomal, non-chromosomal and synthetic DNA sequences.

Any suitable expression vector can be used. For example, prokaryotic cloning vectors include plasmids from E. coli, such as colE1, pCR1, pBR322, pMB9, pUC, pKSM, and RP4. Prokaryotic vectors also include derivatives of phage DNA such as M13 and other filamentous single-stranded DNA phages. An example of a vector useful in yeast is the 2μ plasmid. Suitable vectors for expression in mammalian cells include well-known derivatives of SV-40, adenovirus, retrovirus-derived DNA sequences and shuttle vectors derived from combination of functional mammalian vectors, such as those described above, and functional plasmids and phage DNA.

Additional eukaryotic expression vectors are known in the art (e.g., P. J. Southern and P. Berg, J. Mol. Appl. Genet, 1:327-41 (1982); Subramani et al., Mol. Cell. Biol. 1:854-64 (1981); Kaufmann and Sharp, “Amplification And Expression of Sequences Cotransfected with a Modular Dihydrofolate Reductase Complementary DNA Gene,” J. Mol. Biol. 159:601-21 (1982); Kaufmann and Sharp, Mol. Cell. Biol. 159:601-64 (1982); Scahill et al., “Expression And Characterization Of The Product Of A Human Immune Interferon DNA Gene In Chinese Hamster Ovary Cells,” Proc. Nat'l Acad. Sci. USA 80, 4654-59 (1983); Urlaub and Chasin, Proc. Nat'l Acad. Sci. USA 77:4216-20, (1980).

The expression vectors useful in the present invention contain at least one expression control sequence that is operatively linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof.

Where it is desired to express a gene construct in yeast, a suitable selection gene for use in yeast is the trp1 gene present in the yeast plasmid YRp7. Stinchcomb et al. Nature, 282:39 (1979); Kingsman et al., Gene, 7:141 (1979). The trp1 gene provides a selection marker for a mutant strain of yeast lacking the ability to grow in tryptophan, for example, ATCC No. 44076 or PEP4-1. Jones, Genetics, 85:12(1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

The present invention also provides recombinant host cells containing the expression vectors previously described. Antibodies of the present invention can be expressed in cell lines other than in hybridomas. Nucleic acids, which comprise a sequence encoding a polypeptide according to the invention, can be used for transformation of a suitable mammalian host cell.

Cell lines of particular preference are selected based on high level of expression, constitutive expression of protein of interest and minimal contamination from host proteins. Mammalian cell lines available as hosts for expression are well known in the art and include many immortalized cell lines, such as but not limited to, COS-7 cells, Chinese Hamster Ovary (CHO) cells, Baby Hamster Kidney (BHK) cells and many others including cell lines of lymphoid origin such as lymphoma, myeloma, or hybridoma cells. Suitable additional eukaryotic cells include yeast and other fungi. Useful prokaryotic; hosts include, for example, E. coli, such as E. coli SG-936, E. coli HB 101, E. coli W3110, E. coli X1776, E. coli X2282, E. coli DHI, and E. coli MRCl, Pseudomonas, Bacillus, such as Bacillus subtilis, and Streptomyces.

The recombinant host cells can be used to produce an antibody, or fragment thereof, by culturing the cells under conditions permitting expression of the antibody or antibody fragment and purifying the antibody or antibody fragment from the host cell or medium surrounding die host cell. Targeting of the expressed antibody or fragment for secretion in the recombinant host cells can be facilitated by inserting a signal or secretory leader peptide-encoding sequence (see, Shokri et al., Appl Microbiol Biotechnol. 60:654-64 (2003) Nielsen et al., Prot. Eng. 10:1-6 (1997) and von Heinje et al., Nucl. Acids Res. 14:4683-90 (1986)) at the 5′ end of the antibody-encoding gene of interest. These secretory leader peptide elements can be derived from either prokaryotic or eukaryotic sequences. Accordingly, suitable secretory leader peptides, being amino acids joined to the N-terminal end of antibody chains, are used to direct movement of the antibody chains out of the host cell cytosol for secretion into the medium.

The transformed host cells are cultured by methods known in the art in a liquid medium containing assimilable sources of carbon (carbohydrates such as glucose or lactose), nitrogen (amino acids, peptides, proteins or their degradation products such as peptones, ammonium salts or the like), and inorganic salts (sulfates, phosphates and/or carbonates of sodium, potassium, magnesium and calcium). The medium furthermore contains, for example, growth-promoting substances, such as trace elements, for example iron, zinc, manganese and the like.

Another way to prepare an antibody of the present invention is to express a nucleic acid encoding the antibody in a transgenic animal. Useful transgenic animals, include but are not limited to mice, goats, and rabbits. In an embodiment of the invention, the antibody encoding gene is expressed in the mammary gland of the animal and the antibody is produced in breast milk during lactation.

High affinity anti-IGF-IR antibodies according to the present invention can be isolated from a phage display library displaying human variable domains. In one embodiment, the variable regions are displayed as single chain Fvs (scFvs). In another embodiment, the variable regions are displayed as Fabs. Productively rearranged genes encoding complete variable domains can be obtained from peripheral blood lymphocytes. Alternatively, die variable domains can be partially or completely synthesized. In one embodiment, human V gene segments are combined with synthetic D and J segments. In another embodiment, human CDRs and FWs from different sources are recombined. For example, CDRs can be amplified from human sequences and recombined into consensus human FWs.

Single domain antibodies can be obtained by selecting a V_{H} or a V_{L} domain from a naturally occurring antibody or hybridoma, or selected from a library of V_{H} domains or a library of V_{L} domains. It is understood that amino acid residues that are primary determinants of binding of single domain antibodies can be within Kabat defined CDRs, but may include other residues as well, such as, for example, residues that would otherwise be buried in the V_{H}-V_{L} interface of a V_{H}-V_{L} heterodimer.

In the examples below, over 90% of recovered Fab clones after three rounds of selection were specific to IGF-IR. The binding affinities for IGF-IR of the screened Fabs can be in the nM range, which is as high as many bivalent anti-IGF-IR monoclonal antibodies produced using hybridoma technology.

Antibodies of the present invention also include those for which binding characteristics have been improved by direct mutation, methods of affinity maturation, or chain shuffling. For example, affinity and specificity may be modified or improved by mutating CDRs and screening for antigen binding sites having the desired characteristics (see, e.g., Yang et al., J. Mol. Biol., 254:392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Biol., 226:889-896 (1992)). For example, phage display vectors containing heavy and light chain variable region genes may be propagated in mutator strains of E. coli (see, e.g., Low et al., J. Mol. Biol., 250:359-368 (1996)). These methods of mutagenesis are illustrative of the many methods known to one of skill in the art.

The protein used to identify IGF-IR binding antibodies of the invention is preferably IGF-IR and, more preferably, is the extracellular domain of IGF-IR. The IGF-IR extracellular domain can be free or conjugated to another molecule.

Other examples of IGF-IR specific antibodies include XenoMouse® derived human antibody CP-751871 (Cohen, B. et al, 2005, Clin. Cancer Res. 11:2063-73), humanized antibody EM164 (Maloney, E. K. et al., 2003, Cancer Res. 63:5073-83), humanized antibody h7C10 (Goetsch, L. et al., 2005, Int. J. Cancer 113:316-28), AMG-479 (Amgen) and scFv-Fc-IGF-IR (Sachdev, D. et al., 2003, Cancer Res., 63:627-35).

The antibodies of this invention can be fused to additional amino acid residues. Such amino acid residues can be a peptide tag, perhaps to facilitate isolation.

In other embodiments, IGF-IR antagonists that bind to a ligand of IGF-IR can be used. Examples of such antagonists include, but are not limited to, antibodies that bind to IGF-I or IGF-II and soluble IGF-IR fragments that bind to those ligands.

Another means to block IGF-IR mediated signal transduction is via small molecule inhibitors of IGF-IR. Small molecule refers to small organic compounds, such as heterocycles, peptides, saccharides, steroids, and the like. The small molecule modulators preferably have a molecular weight of less than about 2000 Daltons, preferably less than about 1000 Daltons, and more preferably less than about 500 Daltons. The compounds may be modified to enhance efficacy, stability, pharmaceutical compatibility, and the like. The small molecule inhibitors include but are not limited to small molecules that block the ATP binding domain, substrate binding domain, or kinase domain of receptor tyrosine kinases. In addition to receptor tyrosine kinases, small molecules can be inhibitors of other components of the IGF-IR signal transduction pathway. In another embodiment, a small molecule inhibitor binds to the ligand binding domain of IGF-IR and blocks receptor activation by an IGF-IR ligand.

Small molecule libraries can be screened for inhibitory activity using high-throughput biochemical, enzymatic, or cell based assays. The assays can be formulated to detect the ability of a test compound to inhibit binding of IGF-IR to IGF-IR ligands or substrate IRS-1 or to inhibit the formation of functional receptors from IGF-IR dimers. Small molecule antagonists of IGF-IR include, for example, the insulin-like growth factor-I receptor selective kinase inhibitors NVP-AEW541 (García-Echeverría, C. et al., 2004, Cancer Cell 5:231-9) and NVP-ADW742 (Mitsiades, C. et al., 2004, Cancer Cell 5:221-30), INSM-18 (Insmed Incorporated), which is reported to selectively inhibit IGF-IR and HER2, and the tyrosine kinase inhibitor tryphostins AG1024 and AG1034 (Párrizas, M. et al., 1997, Endocrinology 138:1427-33) which inhibit phosphorylation by blocking substrate binding and have a significantly lower IC₅₀ for inhibition of IFG-IR phorphorylation than for ER phosphorylation. The cyclolignan derivative picropodophyllin (PPP) is another IGF-IR antagonist that inhibits IGF-IR phosphorylation without interfering with ER activity (Girnita, A. et al., 2004, Cancer Res. 64:236-42). Other small molecule IGF-IR antagonists include the benzimidazol derivatives BMS-536924 (Wittman, M. et al., 2005, J. Med. Chem. 48:5639-43) and BMS-554417 (Haluska P. et al., 2006, Cancer Res. 66:362-71), which inhibit IGF-IR and IR almost equipotently. For compounds that inhibit receptors in addition to IGF-IR, it should be noted that IC₅₀ values measured in vitro in direct binding assays may not reflect IC₅₀ values measured ex vivo or in vivo (i.e., in intact cells or organisms). For example, where it is desired to avoid inhibition of IR, a compound that inhibits IR in vitro may not significantly affect the activity of the receptor when used in vivo at a concentration that effectively inhibits IGF-IR.

Antisense oligodeoxynucleotides, antisense RNAs and small inhibitory RNAs (siRNA) provide for targeted degradation of mRNA, thus preventing the translation of proteins. Accordingly, expression of receptor tyrosine kinases and other proteins critical for IGF signaling can be inhibited. The ability of antisense oligonucleotides to suppress gene expression was discovered more than 25 yr ago (Zamecnik and Stephenson, Proc. Natl. Acad. Sci. USA. 75:280-284(1978)). Antisense oligonucleotides base pair with mRNA and pre-mRNAs and can potentially interfere with several steps of RNA processing and message translation, including splicing, polyadenylation, export, stability, and protein translation (Sazani and Kole, J. Clin. Invest. 112:481-486(2003)). However, the two most powerful, and widely used antisense strategies are the degradation of mRNA or pre-mRNA via RNaseH and the alteration of splicing via targeting aberrant splice junctions. RNaseH recognizes DNA/RNA heteroduplexes and cleaves the RNA approximately midway between the 5′ and 3′ ends of the DNA oligonucleotide. Inhibition of IGF-IR by antisense oligonucleotides is exemplified in Wraight, Nat. Biotechnol. 18:521-6.

Innate RNA-mediated mechanisms can regulate mRNA stability, message translation, and chromatin organization (Mello and Conte Nature. 431:338-342 (2004)). Furthermore, exogenously introduced long double-stranded RNA (dsRNA) is an effective tool for gene silencing in a variety of lower organisms. However, in mammals, long dsRNAs elicit highly toxic responses that are related to the effects of viral infection and interferon production (Williams Biochem. Soc. Trans. 25:509-513. (1997)). To avoid this, Elbashir and colleagues (Elbashir et al., Nature. 411:494-498 (2001)) initiated the use of siRNAs composed of 19-mer duplexes with 5′ phosphates and 2 base 3′ overhangs on each strand, which selectively degrade targeted mRNAs upon introduction into cells.

The action of interfering dsRNA in mammals usually involves two enzymatic steps. First, Dicer, an RNase III-type enzyme, cleaves dsRNA to 21-23-mer siRNA segments. Then, RNA-induced silencing complex (RISC) unwinds the RNA duplex, pairs one strand with a complementary region in a cognate mRNA, and initiates, cleavage at a site 10 nucleotides upstream of the 5′ end of the siRNA strand (Hannon Nature. 418:244-251 (2002)). Short, chemically synthesized siRNAs in the 19-22 mer range do not require the Dicer step and can enter the RISC machinery directly. It should be noted that either strand of an RNA duplex can potentially be loaded onto the RISC complex, but the composition of the oligonucleotide can affect the choice of strands. Thus, to attain selective degradation of a particular mRNA target, the duplex should favor loading of the antisense strand component by having relatively weak base pairing at its 5′ end (Khvorova Cell. 115:209-216 (2003)). Exogenous siRNAs can be provided as synthesized oligonucleotides or expressed from plasmid or viral vectors (Paddison and Hannon Curr. Opin. Mol. Ther. 5:217-224 (2003)). In the latter case, precursor molecules are usually expressed as short hairpin RNAs (shRNAs) containing loops of 4-8 nucleotides and stems of 19-30 nucleotides; these are then cleaved by Dicer to form functional siRNAs.

Other means to inhibit IGF-IR mediated signal transduction include, but are not limited to, IGF-I or IGF-II mimetics that bind to but do not activate the receptor, and expression of genes or polynucleotides that reduce IGF-IR levels or activity such as triple helix inhibitors and dominant negative IGF-IR mutants.

According to the invention, modulation of body weight and composition in a mammal is accomplished by administering an therapeutically effective amount of an IGF-IR antagonist. “Therapeutically effective amount” refers to an amount of an IGF-IR antagonist having a body weigh or body composition modulating effect. Therapeutically effective amount also refers to a target serum concentration shown to be effective in modulating body weight or composition. Determining the therapeutically effective amount of an IGF-IR antagonist is within the ordinary skill of the art and requires no more than routine experimentation.

One of skill in the art would understand that dosages and frequency of treatment depend on the tolerance of the individual patient and on the pharmacological and pharmacokinetic properties of IGF-IR antagonist used. To achieve saturable pharmacokinetics the loading dose of an anti-IGF-IR antibody can range, for example, from about 10 to about 1000 mg/m², preferably from about 200 to about 400 mg/m². This can be followed by several additional daily or weekly dosages ranging, for example, from about 200 to about 400 mg/m². (For conversions between mg/kg and mg/m² for humans and other mammals, see Freireich, E. J. et al., 1966, Cancer Chemother. Rep. 50:219-44.) The patient is monitored for side effects and the treatment is stopped when such side effects are severe. Depending on the desired outcome, saturation kinetics may not be desired.

In the present invention, any suitable method or route can be used to administer IGF-IR antagonists of the invention, and optionally, to co-administer anti-obesity drugs or agents. The anti-obesity agent regimens utilized according to the invention, include any regimen believed to be optimally suitable for the treatment of the patient's obese condition. Routes of administration include, for example, oral, intravenous, intraperitoneal, subcutaneous, or intramuscular administration. The dose of antagonist administered depends on numerous factors, including, for example, the type of antagonists, the type and severity of obesity being treated and the route of administration of the antagonists. If should be emphasized, however, that the present invention is not limited to any particular method or route of administration.

It is understood that an IGF-IR antagonist of the invention, where used in a mammal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers can further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active: ingredient after administration to the mammal.

According to the invention, one or more IGF-IR antagonists can be used in combination as well as in combination with other anti-obesity agents or drugs, behavioral modifications, or surgical interventions.

Examples of anti-obesity drugs include lipase inhibitors (i.e., carbohydrate blockers or fat-blockers) such as orlistate (Xenical), cetilistat (ATL-962), and Peptimmune's GT 389-255 that block the bodily absorption of fat, AOD 9604 (hGH 177-191) that increases metabolism and oleoyl-estrone (OE) that induces the wasting of adipose tissue. Orlistat, cetilistat and GT 389-255 are lipase inhibitors, that act by inhibiting the absorption of dietary fats. Orlistat forms a covalent bond with the active serine residue site of gastric and pancreatic lipases, thus preventing triglycerides from being hydrolyzed into absorbable fatty acids and monoglycerides. Cetilistat acts similarly to orlistat, while GT 389-255 is a conjugate of a lipase inhibitor and a fat-binding polymer. The invention of blocking IGF-IR alone or in combination with the lipase inhibitors could be used to reduce obesity as well as a treatment to prevent recurrence of obesity. Another class of ah anti-obesity drug that could be used in combinatorial therapy are drugs that suppress appetite such as sibutramine (Meridia). Sibutramine is thought to work by increasing the activity of certain chemicals, called norepinephrine, serotonin, and to a much lesser extent, dopamine in the brain resulting in satiety and decreased caloric intake. Other drugs that work similarly as sibutramine in suppressing appetite are rimonabant (Acomplia), APD356, Pramlintide/AG137 (Symlin), PYY3-36, AC 162352, oxyntomodulin and TM 30338. Another embodiment of the invention would be a combinatorial therapy that along with blocking the IGF-IR axis, involve manipulation of leptin and/or ghrelin, hormones that help to control satiety and hunger in human physiology. Anti-ghrelin vaccine could be used to manipulate the physiological level, of ghrelin in the body. Metformin (Glucophage) is another drug that could have an effect on obesity. Metformin is used to regulate blood glucose (sugar) levels for treating diabetes type II. It could be used to treat obesity by reducing the amount of glucose absorbed from food through your stomach. In addition to lipase inhibitors and appetite suppressants, many amphetamine products have been FDA-approved for the treatment of obesity, and thus, they could also be used in combinatorial therapy to treat obesity. The list includes phentermine, phendimetrazine, methamphetamine, benzphetamine, and diethylpropion with phentermine being the most popularly prescribed (Stafford R. S., Radley, D. C. Arch Intern. Med. 163:1046-50 (2003)). In certain embodiments, the IGF-IR antagonist with or without other drugs is part of a comprehensive treatment for obesity, including modifications in diet (e.g., hypocaloric), exercise and/or behavioral modification. In other embodiments, the IGF-IR antagonist is part of a treatment that includes surgical intervention. Examples of surgical, intervention include removal of visceral fat, IGF-IR antagonists can also be combined with bariatric surgery (including, for example, gastric bypass, gastric-banding, and vertical gastrectomy) for treatment of morbid obesity.

In a combination therapy, the IGF-IR antagonist is administered before, during, or after commencing therapy with another agent, as well as any combination thereof, i.e., before and during, before and after, during and after, or before, during and after commencing the anti-obesity agent therapy. For example, the IGF-IR antagonist can be administered between 1 and 30 days, preferably 3 and 20 days, more preferably between 5 and 12 days before commencing administration of an anti-obesity drug. In a preferred embodiment of the invention, an anti-obesity agent is administered concurrently with or, more preferably, subsequent to antibody therapy.

The present invention also includes kits for treating or ameliorating obesity comprising a therapeutically effective amount of an IGF-IR antagonist. The kits can further contain any suitable anti-obesity agent for coadministration with the IGF-IR antagonist.

The present IGF-IR antagonists can be used in vivo and in vitro for investigative, or diagnostic methods, which are well known in the art. The diagnostic methods include kits, which contain IGF-IR antagonists of the present invention.

Of course, it is to be understood and expected that variations in the principles of invention herein disclosed can be made by one skilled in the art and it is intended that such modifications are to be included within the scope of the present invention.

The following examples further illustrate the invention, but should not be construed to limit the scope of the invention in any way. Detailed descriptions of conventional methods, such as those employed in the construction of vectors and plasmids, the insertion of genes encoding polypeptides into such vectors and plasmids, the introduction of plasmids into host cells, the expression and determination thereof of genes and gene products, and immunological techniques can be obtained from numerous publications, including Sambrook, J. et al., (1989) Molecular Cloning: A Laboratory Manual, 2^{nd} ed., Cold Spring Harbor Laboratory Press; and Coligan, J. et al. (1994) Current Protocols in Immunology, Wiley & Sons, Incorporated. All references mentioned herein are incorporated by reference in their entirety.

### EXAMPLES

### Example 1

### Selection and Engineering of Anti-Human IGF-IR Monoclonal Antibodies

In order to isolate high affinity antibodies to the human IGF-Ireceptor, recombinant extracellular portion of human IGF-IR (See, Genbank Accession No. NP 000866; Ullrich, A. et al., 1986, EMBO J. 5:2503-12) was used to screen a human naïve (non-immunized) bacteriophage Fab library containing 3.7×10¹⁰ unique clones (de Haard et al., J. Biol. Chem. 274:18218-30 (1999)). Soluble IGF-IR (50 μg/ml) was coated onto tubes and blocked with 3% milk/PBS at 37 degrees for 1 hour. Phage were prepared by growing library stock to log phase culture, rescuing with M13K07 helper phage, and amplifying overnight at 30° C. in 2YTAK culture medium at containing ampicillin and kanamycin selection. The resulting phage preparation was precipitated in 4% PEG/0.5M NaCl and resuspended in 3% milk/PBS. The immobilized receptors were then incubated with phage preparation for 1 hour at room temperature. Afterwards, the tubes were washed 10 times with PBST (PBS containing 0.1% Tween-20) followed by 10 times with PBS. The bound phage were eluted at RT for 10 min with 1 ml of a freshly prepared solution of 100 mM triethylamine. The eluted phage were incubated with 10 ml of mid-log phase TG1 cells at 37° C. for 30 min stationary and 30 min shaking. The infected TG1 cells were pelleted and plated onto several large 2YTAG plates and incubated overnight at 30° C. All colonies that grew on the plates were scraped into 3 to 5 ml of 2YTA medium, mixed with glycerol (final concentration: 10%), aliquoted and stored at −70° C. For second round selection, 100 μl of the phage stock was added to 25 ml of 2YTAG medium and grown to mid-log phase. The culture was rescued with M13K07 helper phage, amplified, precipitated, and used for selection following the procedure described above, but with reduced concentration (5 μg/ml) of IGF-IR immobilized onto tubes and increasing the numbers of washes following the binding process. A total of two rounds of selection were performed.

Individual TG1 clones were picked and grown at 37° C. in 96 well plates and rescued with M13K07 helper phage as described above. The amplified phage preparation was blocked with 1/6 volume of 18% milk/PBS at RT for 1 h and added to Maxi-sorb 96-well microliter plates (Nunc) coated with IGF-IR (1 μg/ml×100 μl). After incubation at RT for 1 h the plates were washed 3 times with PBST and incubated with a mouse anti-M13 phage-HRP conjugate (Amersham Pharmacia Biotech, Piscataway, N.J.). The plates were washed 5 times, TMB peroxidase substrate (KPL, Gaithersburg, Md.) added, and the absorbance at 450 nm read using a microplate reader (Molecular Device, Sunnyvale, Calif.). From 2 rounds of selection, 80% of independent clones were positive for binding to IGF-IR.

The diversity of the anti-IGF-IR Fab clones after the second round of selection was analyzed by restriction enzyme digestion pattern (i.e., DNA fingerprint). The Fab gene insert of individual clones was PCR amplified using primers: PUC19 reverse (5′-AGCGGATAACAATTTCACACAGG-3; SEQ ID NO:31) and fdtet seq (5′-GTCGTCTTTCCAGACGTTAGT-3′; SEQ ID NO:32) which are specific for sequences flanking the unique Fab gene regions within the phage vector. Each amplified product was digested with a frequent-cutting enzyme, BstN I, and analyzed on a 3% agarose gel. A total, of 25 distinct patterns were identified. DNA sequences of representative clones from each digestion pattern were determined by dideoxynucleotide sequencing.

Plasmids from individual clones exhibiting positive binding to IGF-IR and unique DNA profile were used to transform a nonsuppressor E. coli host HB2151. Expression of the Fab fragments in HB2151 was induced by culturing the cells in 2YTA medium containing 1 mM isopropyl-1-thio-β-D-galactopyranoside (IPTG, Sigma) at 30° C. A periplasmic extract of the cells was prepared by resuspending the cell pellet in 25 mM Tris (pH 7.5) containing 20% (w/v) sucrose, 200 mM NaCl, 1 mM EDTA and 0.1 mM PMSF, followed by incubation at 4° C. with gentle shaking for 1 h. After centrifugation at 15,000 rpm for 15 min, the soluble Fab protein was purified from the supernatant by affinity chromatography using Protein G column followed the manufacturer's protocol (Amersham Pharmacia Biotech).

Candidate binding Fab clones were screened for competitive blocking of radiolabeled human IGF-I ligand to immobilized IGF-IR (100 ng/well) coated onto 96 strip-well plates. Fab preparations were diluted and incubated with IGF-IR plates for 0.5-1 hour at room temperature in PBS/0.1% BSA. 40 pM of ¹²⁵I-IGF-I was then added and the plates incubated an additional 90 minutes. Wells were then washed 3 times with ice-cold PBS/0.1% BS A, dried, and then counted in a gamma scintillation counter. Candidates that exhibited greater than 30% inhibition of control radiolabeled, ligand binding in single point assay were selected and in vitro blocking titers determined. Four clones were identified. Of these, only Fab clone 2F8 was shown to inhibit ligand binding by more than 50%, with an IC₅₀ of approximately 200 nM, and it was selected, for conversion to full length IgG1 format. The heavy chain variable; region nucleotide and translated amino acid sequences for 2F8 are provided by SEQ ID NOS:1 and 2, respectively. The nucleotide and translated amino acid sequences of the 2F8 heavy chain engineered as a full length IgG1 are provided by SEQ ID NOS:3 and 4, respectively. Fab 2F8 possesses a lambda light chain constant region. The nucleotide and translated amino acid sequences of the 2F8 light chain variable domain are provided by SEQ ID NOS:5 and 6, respectively. The sequences for foil-length lambda light chain are provided by SEQ ID NOS:7 and 8, respectively. Binding kinetic analysis was performed on 2F8 IgG using a BIAcore unit. This antibody was determined to bind to the IGF-IR with an affinity of 0.5-1 nM (0.5-1×10⁻⁹ M).

In order to improve the affinity of this antibody, a second generation Fab phage library was generated in which the 2F8 heavy chain was conserved and the light chain was varied to a diversity of greater than 10⁸ unique species. This method is termed light chain shuffling and has been used successfully to affinity mature selected antibodies for a given target antigen (Chames et al., J. Immunol. 169:1110-18 (2002)). This library was then screened for binding to the human IGF-IR (10 μg/ml) following procedures as described above, and the panning process repeated an additional three rounds with reduced IGF-IR concentration (2 μg/ml) for enrichment of high affinity binding Fabs. Seven clones were analyzed following round four. All 7 contained the same DNA sequence and restriction digest profile. The single isolated Fab was designated A12 and shown to possess a lambda light chain constant region. The nucleotide and translated amino acid sequences of the 2F8 light chain variable domain are provided by SEQ ID NOS:9 and 10, respectively. The sequences for full-length lambda light chain are provided by SEQ ID NOS:11 and 12, respectively. Comparison of the amino acid sequences of the 2F8 and A12 light chain variable domains revealed 11 amino acid differences. Nine of the differences were within CDRs, with the majority (6 amino acid residues) occurring within CDR3.

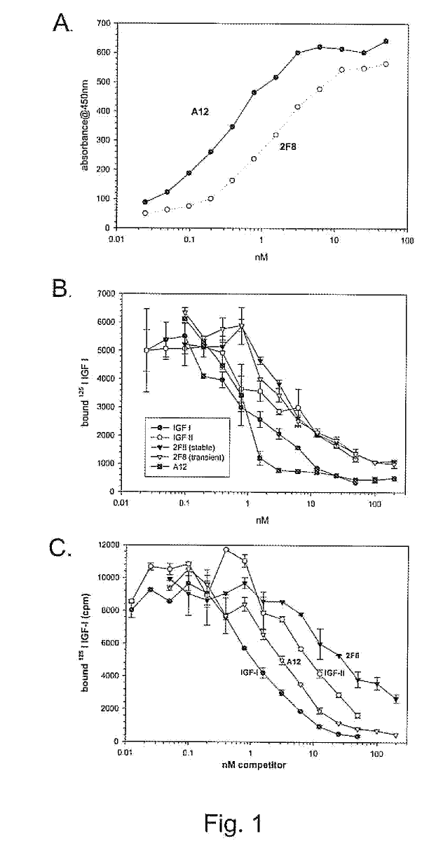

A comparison of the two antibody (full IgG) affinities for human IGF-IR and their ligand blocking activity is shown in Table 3. Binding activity was determined by human IGF-IR-based ELISA (FIG. 1A). Affinity was determined by BIAcore analysis according to manufacturer's specifications (Pharmacia BIACORE 3000). Soluble IGF-IR was immobilized on the sensor chips and antibody binding kinetics determined.

TABLE 3
Antibody binding characteristics

| Antibody | Binding (ED₅₀) | Blocking (EC₅₀) | Affinity |
|---|---|---|---|
| 2F8 | 2.0 nM | 3-6 nM | K_{D} = 6.5 × 10⁻¹⁰ |
| | | | Kₒₙ = 2.8 × 10⁵ |
| | | | K_{off} = 1.8 × 10⁻⁴ |
| A12 | 0.3 nM | 0.6-1 nM | K_{D} = 4.1 × 10⁻¹¹ |
| | | | Kₒₙ = 7.2 × 10⁵ |
| | | | K_{off} = 3.0 × 10⁻⁵ |

A12 also blocked binding of radiolabeled IGF-I ligand to immobilized IGF-IR (FIG. 1B). In this assay, A12 possessed similar blocking activity to cold IGF-I, with an IC₅₀ of approximately 1 nM (0.15 μg/ml), and greater ligand blocking activity than 2F8 or IGF-II (IC₅₀=6 nM).

### Example 2

### Engineering and Expression of Fully Human IgG1 anti-IGF-IR Antibodies from Fab Clones

The DNA sequences encoding the heavy and light chain genes of Fabs 2F8 and A12 were amplified by polymerase chain reaction (PCR) using the Boerhinger Mannheim Expand kit according to manufacturer's instructions. Forward and reverse primers contained sequences for restriction endonuclease sites for cloning into mammalian expression vectors. The recipient vector for the heavy chain contained the entire human gamma 1 constant region cDNA sequence, flanked by a strong eukaryotic promoter and a 3′ polyadenylation sequence. The full-length lambda light chain sequences for 2F8 or A12 were each cloned in to a second vector possessing only the eukaryotic regulatory elements for expression in mammalian cells. A selectable marker was also present on this vector for selection of stable DNA integrants following transfection of the plasmid into mammalian cells. Forward primers were also engineered with sequences encoding a strong mammalian signal peptide sequence for proper secretion of the expressed antibody. Following identification of properly cloned immunoglobulin gene sequences, the DNAs were sequenced and tested for expression in transient transfection. Transient transaction was performed into the COS7 primate cell line using Lipofection, according to manufacturer's specifications. At 24 or 48 hours post-transfection, the expression of full IgG antibody was detected in conditioned culture supernatant by anti-human-Fc binding ELISA. ELISA Plates (96 well) were prepared by coating with 100 ng/well of a goat-anti-human Fc-specific polyclonal antibody (Sigma) and blocked with 5% milk/PBS overnight at 4° C. The plates were then washed 5 times with PBS. Conditioned supernatant was added to wells and incubated for 1.5 hours at room temperature. Bound antibody was detected with a goat anti-human lambda light chain-HRP antibody (Sigma) and visualized with TMB reagents and microplate reader as described above. Large scale preparation of anti-IGF-IR antibodies was achieved by either large scale transient transfection into COS cells, by scale-up of the Lipofection method or by stable transfection into a suitable host cell such as a mouse myeloma cell line (NS0, Sp2/0) or a Chinese hamster ovary cell line (CHO). Plasmid encoding the anti-IGF-IR antibodies were transfected into host cells by electroporation and selected in appropriate drug selection medium for approximately two weeks. Stably selected colonies were screened for antibody expression by anti-Fc ELISA and positive clones expanded into serum free cell culture medium. Antibody production from stably transfected cells was performed in suspension culture in spinner flasks or bioreactors for a period of up to two weeks. Antibody generated by either transient or stable transfection was purified by ProA affinity chromatography (Harlow and Lane. Antibodies. A Laboratory Manual. Gold Spring Harbor Press. 1988), eluted into a neutral buffered saline solution, and quantitated.

### Example 3

### Ligand Blocking Activity of Anti-IGF-IR Monoclonal Antibodies

The anti-IGF-IR antibodies were tested for blocking of radiolabeled ligand binding to native IGF-IR on human tumor cells (FIG. 1C). Assay conditions were performed according to Arteaga and Osborne (Cancer Res. 49:6237-41 (1989)), with minor modifications. MCF7 human breast cancer cells were seeded into 24 well dishes, and cultured overnight. Sub-confluent monolayers were washed 2-3 times in binding buffer (Iscove's Medium/0.1% BSA) and antibody added in binding buffer. After a short incubation with the antibody at room temperature, 40 pM ¹²⁵I-IGF-I (approximately 40,000 cpm/well) was added to each well and incubated for an additional hour with gentle agitation. The wells were then washed three times with ice-cold PBS/0.1% BSA. Monolayers were then lysed with 200 μl 0.5N NaOH and counted in a gamma counter. On human tumor cells, antibody A12 inhibited ligand binding to IGF-IR with an IC₅₀ of 3 nM (0.45 μg/ml). This was slightly lower than the inhibitory activity of cold IGF-I ligand (IC₅₀=1 nM), but better than the inhibitory activity of cold IGF-II (IC₅₀=9 nM). The differences observed for the two IGF ligands can likely be attributed to the slower binding kinetics of IGF-II for the IGF-IR than ligand IGF-I (Jansson et al., J. Biol. Chem. 272:8189-97 (1997). The IC₅₀ for antibody 2F8 was determined to be 30 nM (4.5 μg/ml). Antibody A12 was also shown to be effective in binding to, and inhibiting ligand binding to, endogenous cellular IGF-IR in a variety of other human tumor cell lines from breast, pancreatic, and colorectal tissue (Table 4).

TABLE 4
Inhibitory activity of antibody A12 on IGF-I
binding to different human tumor types

| Cell line | Cell type | Blocking IC₅₀ |
|---|---|---|
| MCF7 | breast | 3 nM |
| T47D | breast | 6 nM |
| OV90 | ovarian | 6 nM |
| BXPC3 | pancreatic | 20 nM |
| HPAC | pancreatic | 10 nM |
| HT-29 | colorectal | 10 nM |
| SK-ES1 | Ewing sarcoma | 2 nM |
| 8226 | myeloma | 20 nM |

### Example 4

### Antibody-Mediated Inhibition of IGF-I Induced Receptor Phosphorylation and Downstream Signaling

To visualize the inhibitory effect of the anti-IGF-IR antibodies on IGF-I signaling, receptor auto-phosphorylation and downstream effector molecule phosphorylation analysis was performed in the presence or absence of antibody A12 or 2F8. The MCF7 human breast cancer cell line was selected for use due to its high IGF-IR density. Cells were plated into 10 cm or 6 well culture dishes and grown to 70-80% confluence. The monolayers were then washed twice in PBS and cultured overnight in serum free defined medium. Anti-IGF-IR antibody was then added in fresh serum-free media (100 nM-10 nM) and incubated with cells for 30 minutes before addition of ligand (10 nM). Cells were incubated with ligand for 10 minutes, then placed on ice and washed with ice-cold PBS. The cells were lysed by the addition of lysis solution (50 mM Tris-HCl, pH 7.4, 150 mM NaCl, 1% TritonX-100, 1 mM EDTA, 1 mM PMSF, 0.5 mM Na₃VO₄, 1 μg/ml leupeptin, 1 μg/ml pepstatin, and 1 μg/ml aprotinin) and the cells scraped into a centrifuge tube kept on ice for 15 minutes. The lysate was then clarified by centrifugation at 4° C. Solubilized IGF-IR was then immunoprecipitated (IP) from the lysate. A12 at 4 μg/ml was incubated with 400 μl of lysate overnight at 4° C. Immune complexes were then precipitated by the addition of ProteinA-agarose beads for 2 hours at 4° C., pelleted, and washed 3 times with lysis buffer. IPs bound to the ProteinA beads were stripped into denaturing gel running buffer. Lysate or IP were processed for denaturing gel electrophoresis and run on a 4-12% acrylamide gel and blotted to nylon or nitrocellulose membrane by western blot according to Towbin et al. (Biotechnology 24:145-9 (1992)). Tyrosine phosphorylated receptor protein was detected using an anti-p-tyrosine antibody (Cell Signaling #9411) and an anti-mouse-HRP secondary antibody. IGF-IR-β was detected with monoclonal antibody C-20 (Santa Cruz Biotech.). Antibodies to detect phospho-Akt was and total Akt were obtained from Pharmingen (BD Biosciences: Cat. #559029, #559028). For MAPK phosphorylation, phospho-p44/42 and total p44/42 was detected with antibodies from Cell Signaling Technology (Beverly, Mass.; Cat. #9101 with #9102). Phospho-IRS-1 and total IRS-1 were detected with #2381 and 2382, respectively, from Cell Signaling. Bands were visualized with the ECL reagent on X-ray film.

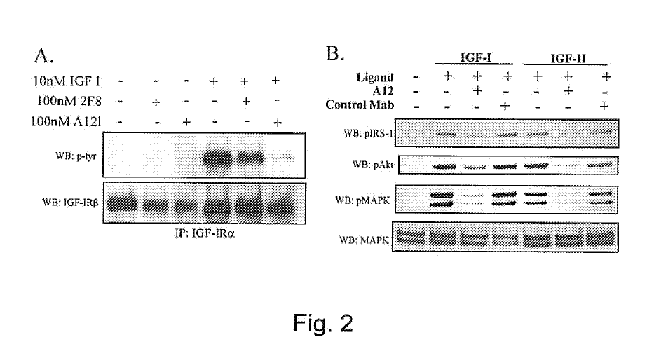

As shown in FIG. 2A, auto-phosphorylation of the IGF-IR in MCF7 cells was arrested following serum deprivation. Addition of either 2F8 or A12 alone did not induce receptor phosphorylation, thereby demonstrating a lack of detectable agonist activity. Upon addition of 10 nM IGF-I, IGF-IR phosphorylation was strongly induced. Antibody 2F8 effected an approximately 50% reduction in IGF-IR phosphorylation, whereas the high affinity antibody A12 nearly completely blocked phosphorylation.

A12 blocks signaling by IGF-I or IGF-II. Western blots were performed oh cells treated with ligand in the presence or absence of A12 pretreatment. As shown in FIG. 2B, the levels of phosphorylated downstream effector molecules IRS-1, Akt, and MAPK in response to both IGF-I and IGF-II were significantly reduced in cells pretreated with A12. The extent of effector molecule inhibition was similar for both ligands, suggesting that A12 is equally proficient at blocking the signaling of both ligands to IGF-IR.

### Example 5

### A12 is a Selective Antagonist of IGF-IR and does not Block the Insulin Receptor

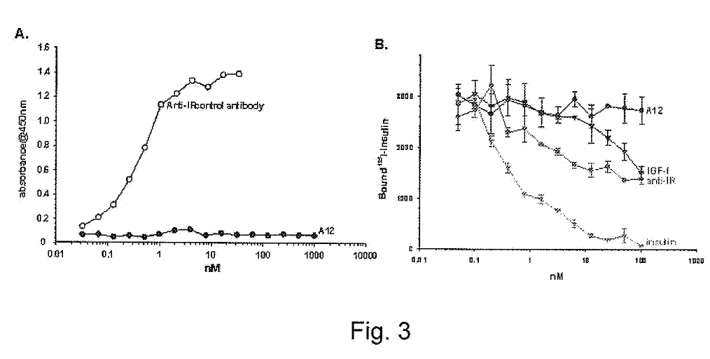

IGF-IR shares considerable structural homology with the insulin receptor (IR). To demonstrate the selectivity of A12 for IGF-IR, the antibody was tested in human IR binding and blocking assays. A12 was titered onto immobilized. ER from a concentration of 1 μM. A commercial anti-human ER antibody was used as a positive control for binding to ER. At a concentration of up to at least 1 μM, there was no detection of bound A12 to IR (FIG. 3A). The ED₅₀ for binding of A12 to human IGF-IR is 0.3 nM, indicating selectivity of A12 for IGF-IR in comparison to ER of greater than 3,000-fold. Accordingly, A12 did not block die binding of insulin to IR (FIG. 3B), even at 100 nM antibody concentration. In this assay, cold insulin effectively competed with an IC₅₀ of approximately 0.5 nM while commercial anti-ER blocking antibody, 47-9, showed modest activity (50% maximal inhibition) and cold IGF-I competed only at high concentrations.

### Example 6

### A12 Recognizes Human and Mouse IGF-IR

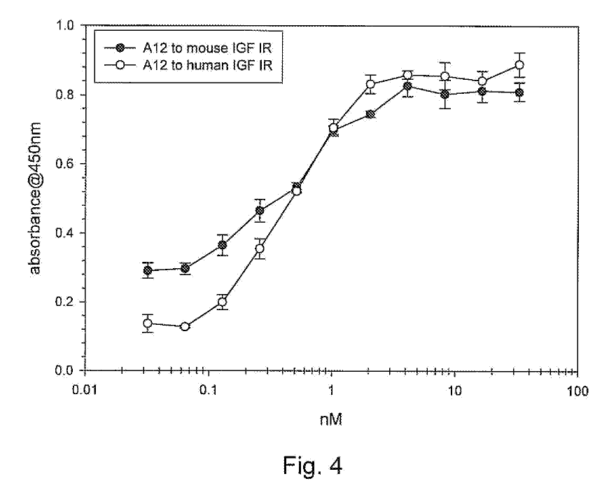

To test for species cross-reactivity to mouse, recombinant mouse IGF-IR (mIGF-IR) was expressed and a binding analysis was performed. This experiment indicated that A12 recognized and bound to immobilized recombinant mIGF-ER in ELISA with an ED₅₀ of 0.3-0.5 nM (FIG. 4). For comparison, the human IGF-IR binding ELISA was repeated with this sample of A12, resulting in an ED₅₀ of 0.3-0.5 nM, consistent with previous results (FIG. 1A). These results suggested that A12 fully cross-reacts with mIGF-IR and binds with similar kinetics to human IGF-IR. Thus A12 can be used in mice to model the effects of blocking IGF-IR in patients.

### Example 7

### A12 Effects on Body Weight in Mice

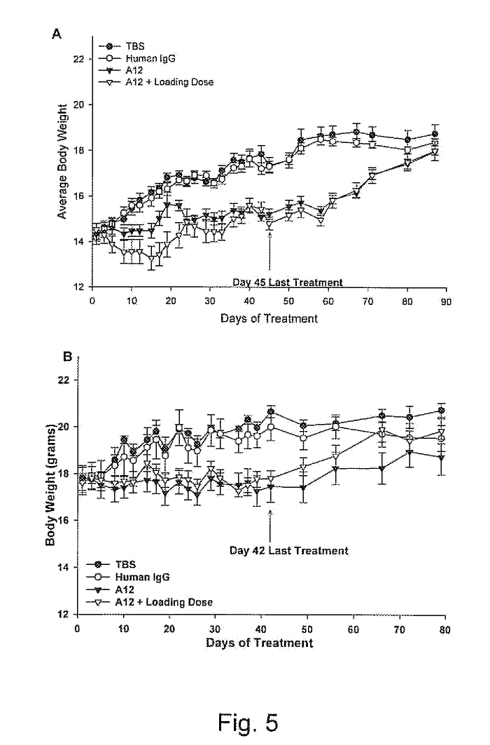

Female Balb/c mice (Charles River Laboratories) and female ob/ob obese mice (Jackson Laboratories, Bar Harbor, Me.) were acclimated to the animal facility for at least one week. Balb/c mice, which normally plateau in body weight at approximately 18 grams, were started on treatment with A12 at approximately 14.5 grams (FIG. 5A). The mice were treated intraperitoneally with either TRIS-buffered saline (TBS), human IgG (Equitech Bio Inc.), or A12 (ImClone Systems Inc. Antibodies were diluted in TBS and administered at 40 mg/kg, Mon-Wed-Fri, with or without a 140 mg/kg loading dose as the first treatment. Body weight was measured 1-2 times per week. Control mice developed normally, increasing in body weight to approximately 18 grams over a 50 day period. During 45 days of A12 treatment, test mice remained at a body weight of about 15 grams, without losing body weight. Treatment was then stopped and A12 treated mice recovered to their normal age related body weight.

In a separate experiment, Balb/c female mice were allowed to mature to a body weight of 18 grams prior to treatment. Control mice in this study continued to increase in body weight to approximately 20 grams (FIG. 5B). A 1.2 again prevented this body weight gain, without causing weight loss. When treatment was stopped after 42 days of treatment, A12 treated mice recovered to their normal age related body weight.

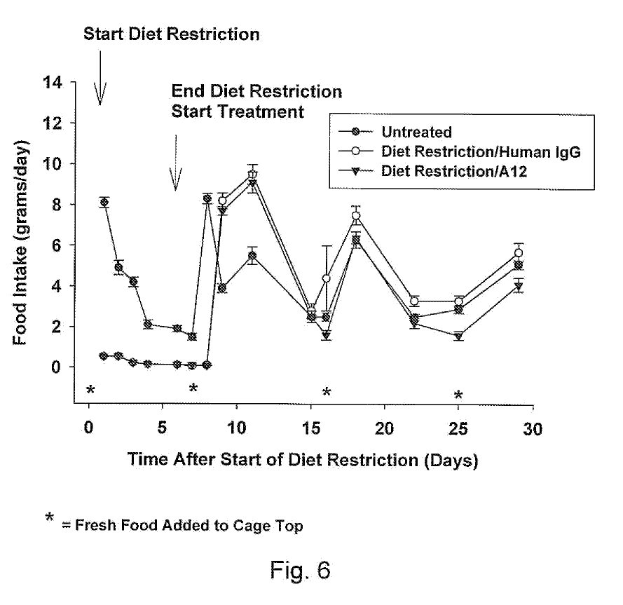

Unwanted weight gain following weight loss in obese individuals was also reduced by treatment with A12. Acclimated ob/ob obese mice (a ieptin deficient obesity model; See, Pelleymounter, M. A, et al., Science 269:540-543 (1995)) were first fed a restricted amount of food (Lab Diet #5001, W.F. Fisher and Son, Inc.) each day for eight days (FIG. 6), then returned ad libitum feeding. Starting about 5 hours prior to return to ad libitum feeding, mice were treated intraperitoneally with either human IgG (Equitech Bio Inc.) or A12 diluted in USP Saline (Braun), at 30 mg/kg, Tuesday and Friday Body weight was measured 1-2 times per week, and daily food intake was estimated in ob/ob mice as the difference in cage top weights between measurements, divided by the number of days between measurements. (FIG. 6).

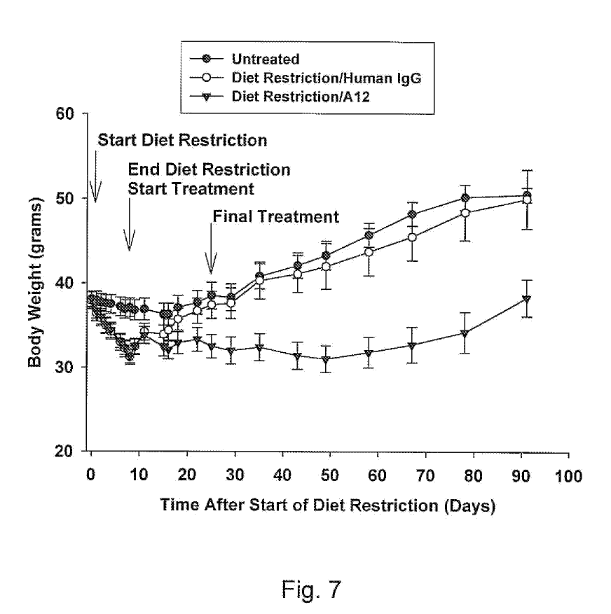

The initial dietary restriction resulted in body weight loss of approximately 18%. Human IgG controls recovered to their normal age related body weight. In contrast, A12 prevented this weight gain without weight loss, compared to the body weight achieved after food restriction (FIG. 7). Moreover, the beneficial effects of A12 on body weight, Were still present for at least 55 days after treatment was stopped.

In an embodiment of the invention, an IGF-IR antagonist promotes weight loss or obesity diminution when used in a monotherapy. In another embodiment, an IGF-IR antagonist promotes weight loss or obesity diminution when combined with a fat-blocking agent. By promoting obesity diminution is meant that administration of an effective amount of antibody, or an effective amount of a combination of an antibody and a fat-blocking agent results in reduced obesity. In a preferred embodiment of the invention, obesity diminution may be observed and continue for a period of at least about 20 days, more preferably at least about 40 days, more preferably at least about 60 days. Obesity diminution can be measured as an average across a group of subjects undergoing a particular treatment regimen, or can be measured by the number of subjects in a treatment group in which obesity diminishes.

### Example 8

### Dose Response Effects of A12 Effects on Increase of Body Weight in Mice

This experiment tested the ability of A12 to i) minimize body weight increase of ob/ob mice following food restriction and ii) to effect weight loss in ob/ob mice fed ad libitum.

Female ob/ob mice (n=47) were allowed to reach approximately 45 grams during an acclimatization period. When the mice-reached a plateau in body weight based on daily measurements over at least a week, food was removed from the cage tops of 36 mice. These food restricted mice were given approximately 0.1-0.2 grams of food per day for 13 days. The remaining mice were given food ad libitum and were considered non-food restricted.

When food restricted mice reached an average weight loss of approximately 22% compared to the initial body weight, these mice were then randomized by body weight into 4 treatment groups: 1) human IgG, 30 mg/kg, ip; 2) A12, 3 mg/kg, ip; 3) A12, 10 mg/kg, ip; and 4) A12, 30 mg/kg, ip. Three hours after receiving their first treatment, animals were, given free access to food. Doses were administered i.p. twice a week for 53 days.

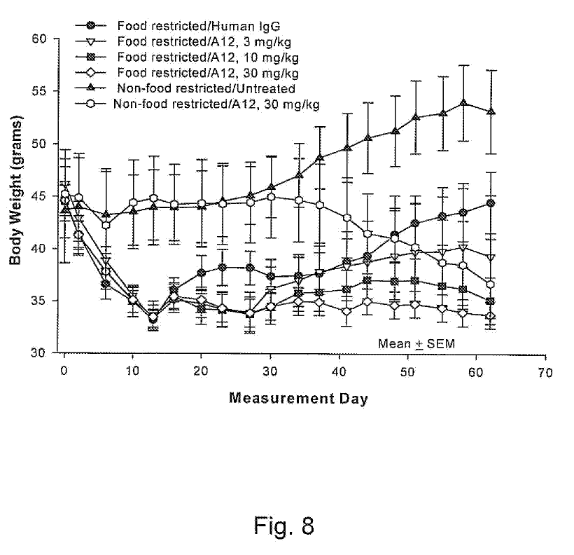

Non-food restricted mice were also randomized by body weight into treatment groups with five mice from this group treated with A12 at 30 mg/kg i.p at the same time as other food restricted groups. The remaining non-food restricted mice were left untreated. Body weight was monitored twice a week throughout the study. Body weight plots again showed that A12 prevented the return to pre-food restriction body weight observed in human IgG treated mice. (FIG. 8) Although food restricted A12 treated mice did not lose weight, non-food restricted A12 treated obese mice lost weight, beginning after approximately 30 days of A12 treatment. Thus inhibition of IGF-IR signaling hot only prevented weight gain, but also induced weight loss in non-dieted obese mice.

1-23. (canceled)

24. A method for modulating body weight in mammals comprising blocking IGF-IR signaling by administering an insulin-like growth factor receptor (IGF-IR) antibody or antibody fragment to a mammal in need thereof.

25. The method of claim 24, wherein said modulating of said body weight results in loss of body weight, maintaining body weight, or minimizing increases in body weight following weight loss in said mammal.

26. The method of claim 24 or 25, wherein the antibody is chimeric, humanized or human.

27. The method of claim 26, wherein the antibody is human.

28. The method of claim 27, wherein the antibody is A12.

29. The method of claim 27, wherein the antibody is 2F8.

30. The method of claim 24 or 25, wherein the antibody or antibody fragment has three heavy chain CDRs corresponding to SEQ ID NO:14, SEQ ID NO:16 and SEQ ID NOS:18, and three light chain CDRs corresponding to SEQ ID NO:26, SEQ ID NO:28 and SEQ ID NO:30.

31. The method of claim 24 or 25, wherein the antibody or antibody fragment has three heavy chain CDRs corresponding to SEQ ID NO:14, SEQ ID NO:16 and SEQ ID NOS:18, and three light chain CDRs corresponding to SEQ ID NO:20, SEQ ID NO:22, and SEQ ID NO:24.

32. The method of claim 24 or 25, wherein the antibody or antibody fragment has a heavy chain variable region of SEQ ID NO:2 and/or a light chain variable region selected from SEQ ID NO:10.

33. The method of claim 24 or 25, wherein the antibody or antibody fragment has a heavy chain variable region of SEQ ID NO:2 and/or a light chain variable region selected from SEQ ID NO:6.

34. The method of claim 24 or 25, wherein the IGF-IR antibody or antibody fragment is administered in an amount ranging from 3-30 mg/kg/day.
